(19) United States
(12) Patent Application Publication
(10) Pub. No.: US 2026/0325739 A1
(43) Pub. Date: Oct. 22, 2026

(54) EAR CANAL CLEANING DEVICE

(71) Applicant: Guangdong Hongkangyi Medical Technology Co., Ltd., Guangdong Province (CN)

(72) Inventors: Yao JIANG, Guangdong Province (CN); Weipeng Jiang, Guangdong Province (CN); Weiqi Jiang, Guangdong Province (CN); Chengjun Gou, Guangdong Province (CN)

(21) Appl. No.: 19/649,108

(22) Filed: Apr. 15, 2026

(30) Foreign Application Priority Data
Apr. 17, 2025 (CN) 2025104845005

(51) Int. Cl. A61M 3/02; A61B 1/00; A61B 1/015; A61B 1/227; A61M 11/02

(52) U.S. Cl. CPC A61M 3/0283; A61B 1/00154; A61B 1/015; A61B 1/227; A61M 3/0258; A61M 11/02; A61M 2205/3653; A61M 2210/0662

(57) ABSTRACT

An ear canal cleaning device, including a base and a handheld component that can be detachably mounted on the base. The handheld component includes a handle and an ear cup. The base integrates a water tank, a control module, and a power supply unit. A viewing tube is disposed within the ear cup, containing an imaging device mounted inside an insertion tube. The imaging device is configured for controlled reciprocating linear motion along a defined axis, enabling users to clearly observe real-time cleaning conditions within the ear canal. The ergonomic design of the ear cup enhances precise visualization of cleaning efficacy. The present invention utilizes air tube, water spray tube, and water suction tube to facilitate comfortable and relaxed body postures during ear canal cleaning while ensuring optimal cleaning performance.

### TECHNICAL FIELD

The present invention relates to medical equipment, particularly to a medical device for cleaning the ear canal.

### BACKGROUND OF THE INVENTION

Existing ear washers clean the ear canal by flushing with water flow, and some solutions also provide an image acquisition function to achieve visual cleaning of the ear canal. However, the ear washers in the prior art have the following technical defects:
1. The focal length of the imaging device used for image acquisition in the prior art ear washers cannot be adjusted, resulting in poor image clarity, which affects the visual effect of the operation and the cleaning effect of the ear canal;
2. The prior art ear washers use water flow for flushing, and after cleaning, the water flow directly drains naturally from the ear canal. Users need to adopt an awkward posture for ear canal cleaning to prevent the drained water from dripping onto themselves; the water temperature for cleaning the ear canal is uncontrollable, causing certain irritation to the user; during and after the ear canal cleaning process, the water remaining in the ear canal needs to be drained by the user themselves or with the help of other tools, affecting the cleaning effect; this results in a poor user experience;
3. The prior art ear washers have complex wire arrangements, are not easy to store, and also lead to a poor user experience.

### BRIEF SUMMARY OF THE INVENTION

The technical problem to be solved by the present invention is to provide an ear canal cleaning device optimally designed from the perspective of user experience, avoiding the shortcomings of the prior art.

The technical problem addressed by the present invention can be solved by adopting the following technical solutions:

A ear canal cleaning device is designed and manufactured, comprising a base and a handheld component that can be removed from and inserted into the base. The handheld component includes a handle that can be held, and an ear cup mounted at the end of the handle and capable of covering the ear. The base includes a water tank, a control module, and a power supply unit disposed therein. An ear insertion tube is provided inside the ear cup, which can be inserted into the ear canal when the ear cup covers the ear. A viewing tube is arranged inside the ear insertion tube, and a imaging device is installed within the viewing tube, which can be controlled to reciprocate along a linear direction.

Specifically, a drive motor controlled by the control module is installed inside the handheld component, and a gear is provided on the output shaft of the drive motor. A external rack gear is provided on the outer wall of the viewing tube. The gear and the external rack gear are engaged, so that the viewing tube reciprocates along a linear direction inside the ear insertion tube under the control of the control module to adjust the focal length of the imaging device.

Specifically, the ear cup includes an ear cup base, at least two ear cup rings, and a connecting ring. The ear insertion tube is fixedly mounted on the ear cup base. the ear cup ring is composed of two conical rings connected by a larger-diameter ring opening, and adjacent ear cup rings are elastically connected to form a bellows-shaped ear cup ring group. An annular clamping groove is machined on the inner wall of the connecting ring, and an annular clamping rib is provided on the outer wall of the ear cup base. The connecting ring is clamped onto the ear cup base by the clamping rib engaging with the clamping groove. The connecting ring is also elastically connected to the ear cup ring at the end of the ear cup ring group, so that the ear cup ring group and the ear cup base with the connecting ring clamped thereon are integrally connected. By means of the elastic connection between the ear cup rings and between the ear cup ring group and the ear cup base, when the ear cup ring group covers the ear, the ear insertion tube fixedly mounted on the ear cup base can adjust the angle of insertion into the ear canal with the deformation of the ear cup ring group.

Specifically, the device further includes an air tube, a water spray tube, and a water suction tube. The ear cup includes an ear cup base, and the ear insertion tube is fixedly mounted on the ear cup base. At least one air pump controlled by the control module and at least one water suction pump controlled by the control module are provided inside the base or the handheld component. At least one water spray pump controlled by the control module is provided inside the water tank. One end of the water spray tube communicates with the interior of the ear insertion tube, and the other end communicates with the water spray pump. Cleaning liquid is sprayed into the ear canal through the ear insertion tube by the water spray pump to clean the ear canal. One end of the air tube communicates with the interior of the ear insertion tube, and the other end communicates with the air pump. Airflow is blown into the ear canal through the ear insertion tube by the air pump to promote the discharge of liquid from the ear canal and timely dry the ear canal. A drain hole is provided at the bottom of the ear cup base. One end of the water suction tube is connected to the drain hole to communicate with the interior of the ear cup base, and the other end communicates with the water suction pump. The used liquid accumulated in the ear cup base from the ear canal is sucked into the water suction tube through the drain hole and discharged by the water suction pump.

Specifically, at least two positioning ribs are provided inside the ear insertion tube, and rib grooves are correspondingly provided on the wall of the viewing tube. Thus, the viewing tube is mounted inside the ear insertion tube by means of the positioning ribs and rib grooves and can reciprocate along a linear direction, forming a separated gap between the viewing tube and the ear insertion tube. One end of the water spray tube communicating with the interior of the ear insertion tube is connected to one gap for spraying water from the ear insertion tube into the ear canal for flushing. One end of the air tube communicating with the interior of the ear insertion tube is connected to another gap for spraying air from the ear insertion tube into the ear canal.

A first separating connector is mounted at the end of the ear insertion tube. The first separating connector is provided with a viewing channel, and a water channel and an air channel are arranged around the viewing channel. The axis of the viewing channel is collinear with the axis of the ear insertion tube. The front end of the water channel faces one gap, and the rear end of the water channel is connected to the end of the water spray tube communicating with the interior of the ear insertion tube. The front end of the air channel faces the other gap, and the rear end of the air channel is connected to the end of the air tube communicating with the interior of the ear insertion tube.

Specifically, at least two positioning ribs are provided inside the ear insertion tube, and grooves are correspondingly provided on the wall of the viewfinder tube. Thus, the viewfinder tube is mounted inside the ear insertion tube by means of the ribs and grooves and can reciprocate along a linear direction, forming a separated gap between the viewfinder tube and the ear insertion tube. Both the end of the water spray tube communicating with the interior of the ear insertion tube and the end of the air tube communicating with the interior of the ear insertion tube are connected to the same gap, so that water, air, or both are sprayed into the ear canal from the ear insertion tube through the same gap.

A second separating connector is mounted at the end of the ear insertion tube. The second separating connector is provided with a viewing channel, at least one discharge channel arranged at the front end around the viewing channel, and a water spray channel and an air jet channel arranged at the rear end around the viewing channel. The water spray channel and the air jet channel together communicate with the same discharge channel. The axis of the viewing channel is collinear with the axis of the ear insertion tube. The front end of the discharge channel faces the gap between the viewing tube and the ear insertion tube. The rear end of the water spray channel is connected to the end of the water spray tube communicating with the interior of the ear insertion tube, and the rear end of the air jet channel is connected to the end of the air tube communicating with the interior of the ear insertion tube.

Specifically, the handle is tubular, and at least one pipeline separating ring is provided inside the handle. The outer ring wall of the pipeline separating ring is adapted to the inner wall of the handle. Inside the pipeline separating ring, an air tube separating ring for passing the air tube, a water spray tube separating ring for passing the water spray tube, a water suction tube separating ring for passing the water suction tube, and a wire separating ring for passing wires are separated by isolation bars and a separating outer ring.

The air tube separating ring, the water spray tube separating ring, and the water suction tube separating ring are all annular, with their centers collinear, and are arranged in the middle of the pipeline separating ring, so that a wire separating ring is formed between these three rings and the separating outer ring of the pipeline separating ring.

Specifically, the ear canal cleaning device further includes at least one air path heating device, which comprises an air path channel tube and an air path heating wire wound around the outside of the air path channel tube. The air path heating wire is electrically connected to the control module and controlled by the control module to adjust its heat output. The air tube includes at least two sub-air tubes, and both ends of the air path channel tube are connected between and communicate with two adjacent sub-air tubes, so that the air path heating device is installed within the pipeline of the air tube pipeline. Controlled by the control module, the air path heating wire heats the air flowing through the air path channel tube.

Specifically, the water tank includes a clean water tank for storing cleaning water for flushing the ear canal and a wastewater tank for storing wastewater sucked back by the water suction tube after flushing. The water spray pump pumps water from the clean water tank to the water spray tube, and the water suction pump draws water from the water suction tube to the wastewater tank. A water path heating wire is installed inside the clean water tank, which is electrically connected to the control module and controlled by the control module to adjust its heat output. Controlled by the control module, the water path heating wire heats the cleaning water pumped into the water spray tube.

The ear canal cleaning device further includes a liquid filtration and purification device installed inside the base. The liquid filtration and purification device communicates with the clean water tank and the wastewater tank, and can filter and purify the liquid in the wastewater tank into cleaning water suitable for cleaning the ear canal, and discharge the cleaning water into the clean water tank.

Specifically, the top of the base is provided with a sunken wire accommodating cavity with a cylindrical surrounding wall. A wire winding base is provided in the middle of the wire accommodating cavity. The top of the wire winding base is provided with a sunken handle cavity with a cylindrical surrounding wall. The side wall of the wire winding base is provided with a wire outlet, which is a sunken notch penetrating the side wall of the wire winding base. A wire winding cavity is formed between the inner wall of the wire accommodating cavity and the outer wall of the wire winding base. Wires for connecting components inside the base and components inside the handheld component are led out of the base from the handle cavity. When the handheld component is inserted into the base, the wires enter the wire winding cavity from the wire outlet and are wound around the outer wall of the wire winding base, and the handle of the handheld component is inserted into the handle cavity of the wire winding base. The top edge of the wire winding base extends outward to form a wire-blocking top eave, which prevents the wires wound around the wire winding base from disengaging from the top of the wire winding base.

Compared with the prior art, the technical effects of the "ear canal cleaning device" of the present invention are as follows:
1. The imaging device of the present invention can be controlled to reciprocate, thereby adjusting the focal length of the imaging device, allowing the user to clearly observe the cleaning condition inside the ear canal.
2. The ear cup structure of the present invention enables the angle of the ear insertion tube within the ear canal to be adjustable, thereby making the framing angle of the imaging device adjustable, which is more conducive for the user to accurately observe the cleaning condition and effect of the ear canal.
3. By providing an air tube, a water spray tube, and a water suction tube, as well as related structures involving them, the present invention enables timely recovery of the water flow used for ear canal cleaning, prevents undischarged water from remaining in the ear canal during and after the cleaning process, ensures timely drying of the ear canal, allows controllable adjustment of the temperature of the flushing water flow and air flow, enables the user to perform ear canal cleaning in a comfortable and relaxed physical posture, ensures the ear canal cleaning effect, and enhances the user experience.
4. Through the partition connectors and line separator rings, the present invention enables systematic, safe, and reliable arrangement of various wires and pipelines in the ear canal cleaning device, prevents adverse mutual influences caused by pipeline tangling, and ensures the operational stability of the device.
5. Through the wire winding base and its related structures, the present invention makes the ear canal cleaning device easier to store and place in daily use, thereby improving the user's experience.

### BRIEF DESCRIPTION OF THE DRAWINGS

FIG. 1 is an axonometric projection schematic diagram of the preferred embodiment of the invention "ear canal cleaning device";

FIG. 2 is a partial cross-sectional schematic diagram of the side orthographic projection of the preferred embodiment of the present invention;

FIG. 3 is an axonometric projection schematic diagram of the base of the preferred embodiment of the present invention;

FIG. 4 is a front orthographic projection schematic diagram of the handheld component of the preferred embodiment of the present invention;

FIG. 5 is an axonometric projection schematic diagram of the disassembled state of the handheld component of the preferred embodiment of the present invention;

FIG. 6 is an axonometric projection schematic diagram of the ear insertion tube of the preferred embodiment of the present invention;

FIG. 7 is a front orthographic projection schematic diagram of the ear insertion tube of the preferred embodiment of the present invention;

FIG. 8 is a front orthographic projection schematic diagram of the pipeline separation ring of the preferred embodiment of the present invention;

FIG. 9 is a front orthographic projection schematic diagram of the air path heating device of the preferred embodiment of the present invention.

### DETAILED DESCRIPTION OF THE INVENTION

The following is a detailed description with reference to the embodiments shown in the drawings.

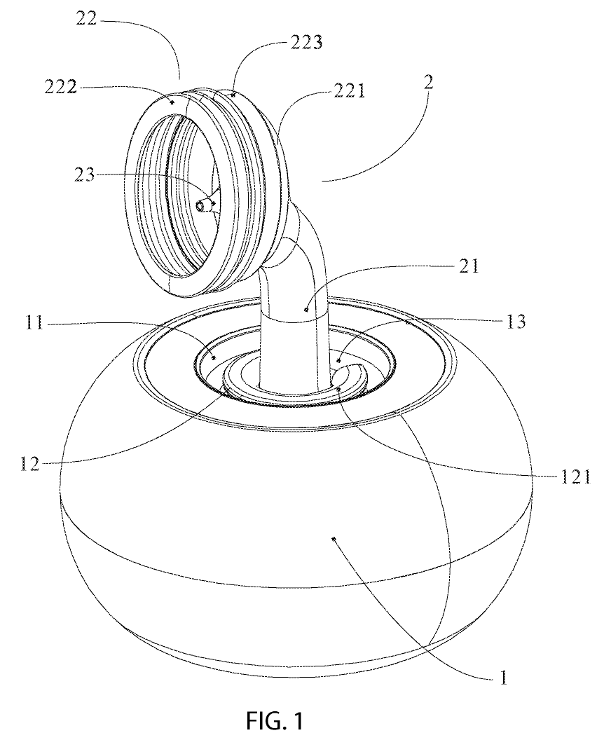

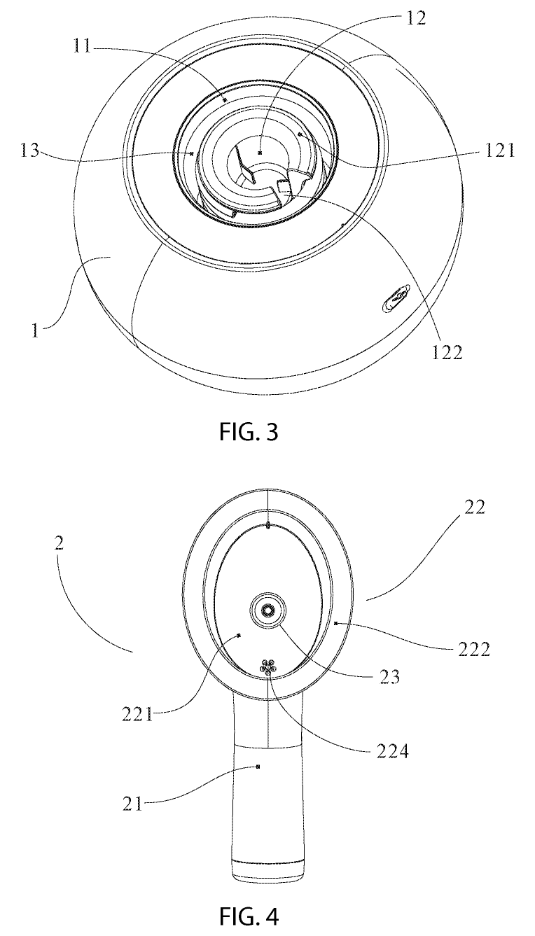

The present invention provides an ear canal cleaning device, as shown in FIGS. 1-5, comprising a base 1 and a handheld component 2 that can be removed from and inserted into the base 1. The handheld component 2 includes a handle 21 that can be held, and an ear cup 22 mounted at the end of the handle 21 and capable of covering the ear. The base 1 includes a water tank (not shown), a control module (not shown), and a power supply unit (not shown) disposed therein. In the preferred embodiment of the present invention, the control module and the power supply unit shall be separated from the water tank with necessary waterproof structural treatments. An ear insertion tube 23 is provided inside the ear cup 22, which can be inserted into the ear canal when the ear cup 22 covers the ear. A viewing tube 24 is arranged inside the ear insertion tube 23, and an imaging device (not shown) is installed within the viewing tube 24, which can be controlled to reciprocate along a linear direction to adjust the focal length; the linear direction may refer to the axial direction of the ear insertion tube 23. In the preferred embodiment of the present invention, the imaging device uses a wireless signal transmission method to transmit collected audio-visual data to a mobile phone or other dedicated wireless audio-visual playback devices via Bluetooth or a wireless network. The viewing tube 24 may also be provided with a lighting device, such as an LED bead, to provide illumination for the imaging device. The imaging device of the present invention can be controlled to reciprocate, thereby adjusting the focal length of the imaging device, allowing the user to clearly observe the cleaning condition of the ear canal.

In the preferred embodiment of the present invention, as shown in FIG. 5, to enable the imaging device to reciprocate under control, a drive motor (not shown) controlled by the control module is installed inside the handheld component 2, and a gear 25 is provided on the output shaft of the drive motor. A external rack gear is provided on the outer wall of the viewing tube 24. The gear 25 and the external rack gear are engaged, so that the viewing tube 24 reciprocates along a linear direction inside the ear insertion tube 23 under the control of the control module to adjust the focal length of the imaging device; the linear direction may refer to the axial direction of the ear insertion tube 23.

In the preferred embodiment of the present invention, as shown in FIG. 5, the handle 21 is formed by connecting two handle shells, and the bottom of the handle 21 is capped by a cover 212.

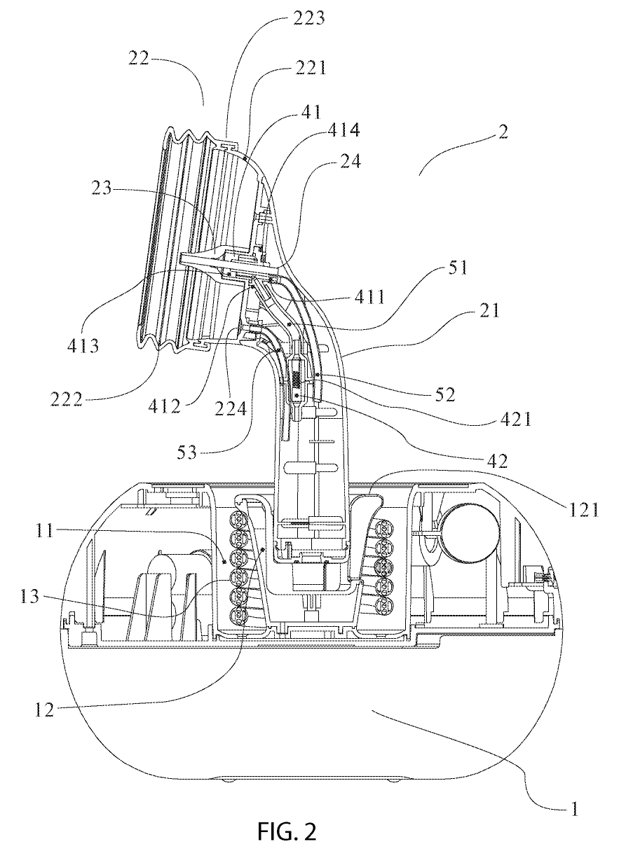

As shown in FIGS. 1 and 2, the preferred embodiment of the invention, the ear cup 22 includes an ear cup base 221, at least two ear cup rings 222, and a connecting ring 223. The ear insertion tube 23 is fixedly mounted on the ear cup base 221. The ear cup ring 222 is composed of two conical rings connected by a larger-diameter ring opening, and adjacent ear cup rings 222 are elastically connected to form a bellows-shaped ear cup ring group. As shown in FIG. 2, an annular clamping groove is machined on the inner wall of the connecting ring 223; an annular clamping rib is provided on the outer wall of the ear cup base 221; the connecting ring 223 is clamped onto the ear cup base 221 by the clamping rib engaging with the clamping groove. The connecting ring 223 is also elastically connected to the ear cup ring 222 at the end of the ear cup ring group, so that the ear cup ring group and the ear cup base 221 with the connecting ring 223 clamped thereon are integrally connected. By means of the elastic connection between the ear cup rings 222 and between the ear cup ring group and the ear cup base 221, when the ear cup ring group covers the ear, the ear insertion tube 23 fixedly mounted on the ear cup base 221 can adjust the angle of insertion into the ear canal with the deformation of the ear cup ring group. The structure of the ear cup 22 of the present invention enables the angle of the ear insertion tube 23 in the ear canal to be adjustable, thereby making the viewing angle of the imaging device adjustable, which is more beneficial for the user to accurately observe the cleaning condition and effect of the ear canal.

In the preferred embodiment of the present invention, as shown in FIG. 2, the ear canal cleaning device further includes an air tube 51, a water spray tube 52, and a water suction tube 53. The ear cup 22 includes an ear cup base 221. The ear insertion tube 23 is fixedly mounted on the ear cup base 221. At least one air pump controlled by the control module and at least one water suction pump controlled by the control module are provided inside the base 1 or the handheld component 2. At least one water spray pump controlled by the control module is provided inside the water tank. One end of the water spray tube 52 communicates with the interior of the ear insertion tube 23, and the other end communicates with the water spray pump. Cleaning liquid is sprayed into the ear canal through the ear insertion tube 23 by the water spray pump to clean the ear canal. One end of the air tube 51 communicates with the interior of the ear insertion tube 23, and the other end communicates with the air pump. Airflow is blown into the ear canal through the ear insertion tube 23 by the air pump to promote the discharge of liquid from the ear canal and timely dry the ear canal.

A drain hole 224 is provided at the bottom of the ear cup base 221. One end of the water suction tube 53 is connected to the drain hole 224 to communicate with the interior of the ear cup base 221, and the other end communicates with the water suction pump. The used liquid accumulated in the ear cup base 221 from the ear canal is sucked into the water suction tube 53 through the drain hole 224 and discharged by the water suction pump. By providing the air tube 51, the water spray tube 52, and the water suction tube 53, and their related structures, the present invention enables the cleaning water flow of the ear canal to be recycled in time, prevents undischarged water from remaining in the ear canal during and after the cleaning process, ensures timely drying of the ear canal, allows the user to perform ear canal cleaning in a comfortable and relaxed posture, ensures the ear canal cleaning effect, and improves the user experience.

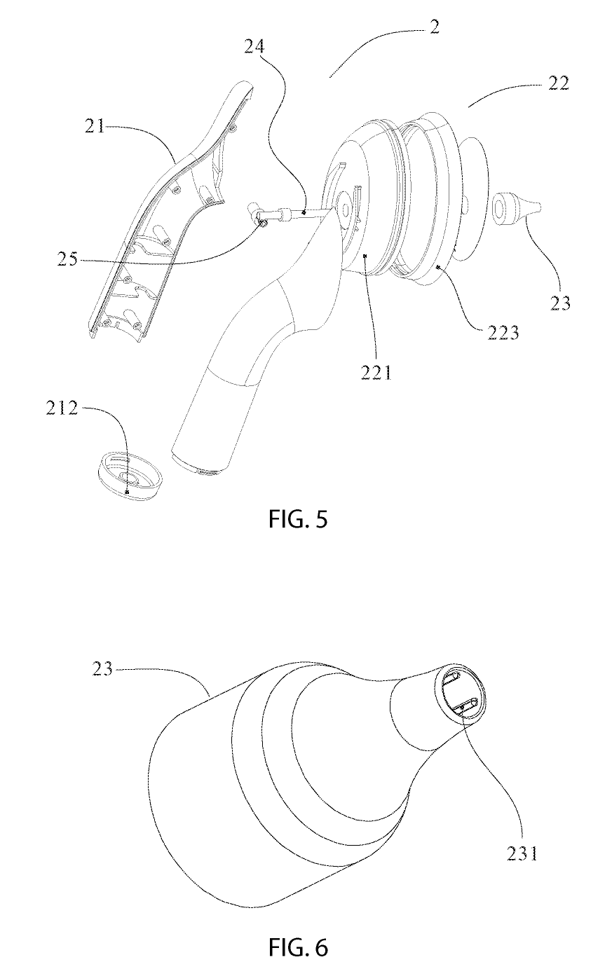

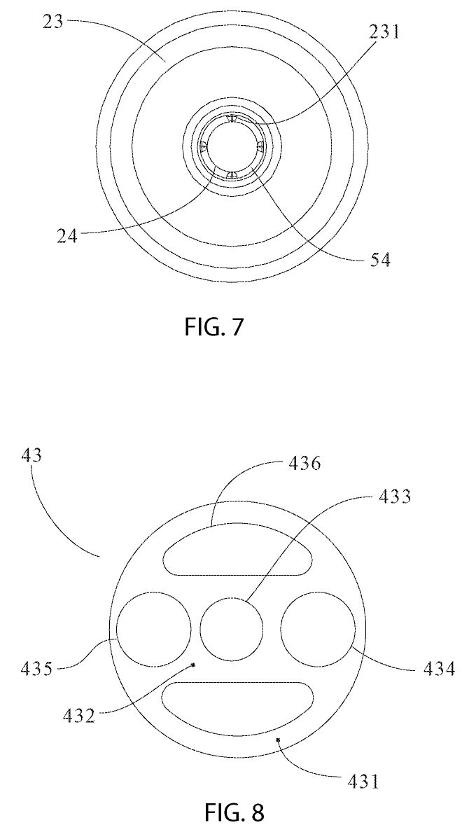

In the preferred embodiment of the present invention, as shown in FIGS. 2, 6 and 7, at least two positioning ribs 231 are provided inside the ear insertion tube 23, and rib grooves are correspondingly provided on the wall of the viewing tube 24. Thus, the viewing tube 24 is mounted inside the ear insertion tube 23 by means of the positioning ribs 231 and rib grooves and can reciprocate along a linear direction, forming a separated gap 54 between the viewing tube 24 and the ear insertion tube 23. Both the end of the water spray tube 52 communicating with the interior of the ear insertion tube 23 and the end of the air tube 51 communicating with the interior of the ear insertion tube 23 are connected to the same gap 54, so that water, air, or both are sprayed into the ear canal from the ear insertion tube 23 through the same gap 54.

As shown in FIG. 2, a second separating connector 41 is mounted at the end of the ear insertion tube 23. The second separating connector 41 is provided with a viewing channel 414, at least one discharge channel 413 arranged at the front end around the viewing channel 414, and a water spray channel 411 and an air jet channel 412 arranged at the rear end around the viewing channel 414. The water spray channel 411 and the air jet channel 412 together communicate with the same discharge channel 413. The axis of the viewing channel 414 is collinear with the axis of the ear insertion tube 23. The front end of the discharge channel 413 faces the gap 54 between the viewing tube 24 and the ear insertion tube 23. The rear end of the water spray channel 411 is connected to the end of the water spray tube 52 communicating with the interior of the ear insertion tube 23; the rear end of the air jet channel 412 is connected to the end of the air tube 51 communicating with the interior of the ear insertion tube 23.

The above structure of the preferred embodiment of the present invention effectively and orderly isolates the water and air transmission channels in a limited space, preventing mutual interference caused by pipeline entanglement. The above structure of the preferred embodiment of the present invention can also be implemented by the following scheme:

At least two positioning ribs 231 are provided inside the ear insertion tube 23, and rib grooves are correspondingly provided on the wall of the viewing tube 24. Thus, the viewing tube 24 is mounted inside the ear insertion tube 23 by means of the positioning ribs and rib grooves and can reciprocate along a linear direction, forming a separated gap 54 between the viewing tube 24 and the ear insertion tube 23. One end of the water spray tube 52 communicating with the interior of the ear insertion tube 23 is connected to one gap 54 for spraying water from the ear insertion tube 23 into the ear canal for flushing. One end of the air tube 51 communicating with the interior of the ear insertion tube 23 is connected to another gap 54 for spraying air from the ear insertion tube 23 into the ear canal.

A first separating connector is mounted at the end of the ear insertion tube. The first separating connector is provided with a viewing channel, and a water channel and an air channel are arranged around the viewing channel. The axis of the viewing channel is collinear with the axis of the ear insertion tube. The front end of the water channel faces one gap 54, and the rear end of the water channel is connected to the end of the water spray tube communicating with the interior of the ear insertion tube. The front end of the air channel faces the other gap 54, and the rear end of the air channel is connected to the end of the air tube communicating with the interior of the ear insertion tube.

In the preferred embodiment of the present invention, as shown in FIG. 8, the handle 21 is tubular, and at least one pipeline separation ring 43 is provided inside the handle 21. The pipeline separation ring 43 may be disposed at any position inside the handle 21 for the purpose of separating pipelines to avoid mutual interference. The installation position of the pipeline separation ring 43 in the handle 21 is not shown in the drawings. The outer ring wall of the pipeline separation ring 43 is adapted to the inner wall of the handle 21. Inside the pipeline separation ring 43, an air tube separation ring 433 for passing the air tube 51, a water spray tube separation ring 434 for passing the water spray tube 52, a water suction tube separation ring 435 for passing the water suction tube 53, and a wire separation ring 436 for passing wires are separated by an isolation bar 432 and a separation outer ring 431.

In the preferred embodiment of the present invention, as shown in FIG. 8, the air tube separation ring 433, the water spray tube separation ring 434, and the water suction tube separation ring 435 are all annular, with their centers collinear, and are arranged in the middle of the pipeline separation ring 43, so that a wire separation ring 436 is formed between these three rings and the separation outer ring 431 of the pipeline separation ring 43. The three rings refer to the air tube separation ring 433, the water spray tube separation ring 434, and the water suction tube separation ring 435.

The present invention uses the first separating connector, and the second separating connector 41 and the pipeline separation ring 43 to orderly, safely, and reliably lay various wires in the ear canal cleaning device, prevent adverse mutual interference caused by pipeline entanglement, and ensure the operational stability of the device.

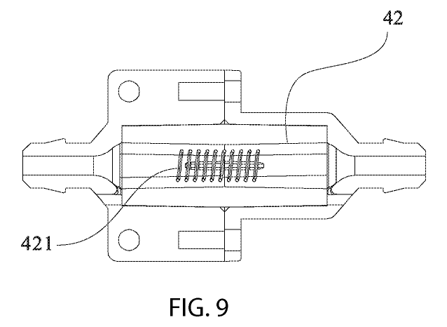

In the preferred embodiment of the present invention, as shown in FIGS. 2 and 9, at least one air path heating device 42 is further included. The air path heating device 42 includes an air path channel tube and an air path heating wire 421 wound around the outside of the air path channel tube. The air path heating wire 421 is electrically connected to the control module and controlled by the control module to adjust its heat output. The air tube 51 includes at least two sub-air tubes, and both ends of the air path channel tube are connected between and communicate with two adjacent sub-air tubes, so that the air path heating device is installed within the pipeline of the air tube 51. Controlled by the control module, the air path heating wire 421 heats the air flowing through the air path channel tube.

In the preferred embodiment of the present invention, the water tank includes a clean water tank for storing cleaning water for flushing the ear canal and a wastewater tank for storing wastewater sucked back by the water suction tube after flushing. The water spray pump pumps water from the clean water tank to the water spray tube 52. The water suction pump draws water from the water suction tube 53 to the wastewater tank. A water path heating wire is installed inside the clean water tank, which is electrically connected to the control module and controlled by the control module to adjust its heat output. Controlled by the control module, the water path heating wire heats the cleaning water pumped into the water spray tube 52.

The present invention enables the temperature of the flushing water flow and air flow to be adjustably controlled, allowing the user to perform ear canal cleaning in a comfortable and relaxed posture, ensuring the ear canal cleaning effect, and improving the user experience.

In the preferred embodiment of the present invention, the ear canal cleaning device further includes a liquid filtration and purification device installed inside the base. The liquid filtration and purification device communicates with the clean water tank and the wastewater tank, and can filter and purify the liquid in the wastewater tank into cleaning water suitable for cleaning the ear canal, and discharge the cleaning water into the clean water tank.

In the preferred embodiment of the present invention, as shown in FIGS. 1-3, the top of the base 1 is provided with a sunken wire accommodating cavity 11 with a cylindrical surrounding wall. A wire winding base 12 is provided in the middle of the wire accommodating cavity 11. The top of the wire winding base 12 is provided with a sunken handle cavity with a cylindrical surrounding wall. The side wall of the wire winding base is provided with a wire outlet 122, which is a sunken notch penetrating the side wall of the wire winding base 12. A wire winding cavity is formed between the inner wall of the wire accommodating cavity 11 and the outer wall of the wire winding base 12 for leading out wires 13 connecting components inside the base 1 and components inside the handheld component 2 from the handle cavity. The wires 13 include wires for electrically connecting electrical components inside the base 1 and the handheld component 2, as well as the air tube 51, the water spray tube 52, and the water suction tube 53; between the base 1 and the handheld component 2, the wires, the air tube 51, the water spray tube 52, and the water suction tube 53 may be independent of each other or bound together without mutual interference. When the handheld component 2 is inserted into the base 1, the wires 13 enter the wire winding cavity from the wire outlet 133 and are wound around the outer wall of the wire winding base 12, and the handle 21 of the handheld component 2 is inserted into the handle cavity of the wire winding base. The top edge of the wire winding base extends outward to form a wire-blocking top eave 121, which prevents the wires 13 wound around the wire winding base 12 from disengaging from the top of the wire winding base 12. The present invention makes the ear canal cleaning device easier to store and display in daily use through the wire winding base 12 and its related structures, thereby enhancing the user experience.

1. An ear canal cleaning device, comprising: a base, and a handheld component that can be removed from and inserted into the base; the handheld component includes a handle that can be held and an ear cup mounted at an end of the handle and capable of covering the ear; the base includes a water tank, a control module, and a power supply unit; wherein,
an ear insertion tube is provided inside the ear cup, which can be inserted into the ear canal when the ear cup covers the ear;
a viewing tube is arranged inside the ear insertion tube, and an imaging device is installed within the viewing tube; which can be controlled to reciprocate along a linear direction.

2. The ear canal cleaning device according to claim 1, wherein,
a drive motor controlled by the control module is installed inside the handheld component; and a gear is provided on an output shaft of the drive motor;an external rack gear is provided on the outer wall of the viewing tube; the gear and external rack gear are engaged so that the viewing tube reciprocates along a linear direction inside the ear insertion tube under control of the control module to adjust focal length of the imaging device.

3. The ear canal cleaning device according to claim 1, wherein,
the ear cup includes an ear cup base, at least two ear cup rings, and a connecting ring;
the ear insertion tube is fixedly mounted on the ear cup base;
the ear cup ring is composed of two conical rings connected by a larger-diameter ring opening, and adjacent ear cup rings are elastically connected to form a bellows-shaped ear cup ring group;
an annular clamping groove is machined on the inner wall of the connecting ring, and an annular clamping rib is provided on the outer wall of the ear cup base; the connecting ring is clamped onto the ear cup base by the clamping rib engaging with the clamping groove; the connecting ring is also elastically connected to the ear cup ring at the end of the ear cup ring group, so that the ear cup ring group and the ear cup base with the connecting ring clamped thereon are integrally connected;
by means of the elastic connection between the ear cup rings and between the ear cup ring group and the ear cup base, when the ear cup ring group covers the ear, the ear insertion tube fixedly mounted on the ear cup base can adjust the angle of insertion into the ear canal with the deformation of the ear cup ring group.

4. The ear canal cleaning device according to claim 1, further comprising: an air tube, a water spray tube, and a water suction tube;
the ear cup includes the ear cup base, and the ear insertion tube is fixedly mounted on the ear cup base;
at least one air pump controlled by the control module and at least a water suction pump controlled by the control module are provided inside the base or handheld component;
at least one water spray pump controlled by the control module is provided inside the water tank;
one end of the water spray tube communicates with the interior of the ear insertion tube, and the other end communicates with the water spray pump, cleaning liquid is sprayed into the ear canal through the ear insertion tube by the water spray pump to clean the ear canal;
one end of the air tube communicates with the interior of the ear insertion tube, and the other end communicates with the air pump, airflow is blown into the ear canal through the ear insertion tube by the air pump to promote the discharge of liquid from the ear canal and timely dry the ear canal;
a drain hole is provided at the bottom of the ear cup base; one end of the water suction tube is connected to the drain hole to communicate with the interior of the ear cup base, and the other end communicates with the water suction pump, the used liquid accumulated in the ear cup base from the ear canal is sucked into the water suction tube through the drain hole and discharged by the water suction pump.

5. The ear canal cleaning device according to claim 4, wherein,
at least two positioning ribs are provided inside the ear insertion tube;and rib grooves are correspondingly provided on the wall of the viewing tube; thus the viewing tube is mounted inside the ear insertion tube by means of the positioning ribs and rib grooves and can reciprocate along a linear direction, forming a separated gap between the viewing tube and the ear insertion tube;
both the end of the water spray tube communicating with the interior of the ear insertion tube and the end of the air tube communicating with the interior of the ear insertion tube are connected to the same gap, so that water, air, or both are sprayed into the ear canal from the ear insertion tube through the same gap.

6. The ear canal cleaning device according to claim 5, wherein,
a second separating connector is mounted at the end of the ear insertion tube;
the second separating connector is provided with a viewing channel, at least one discharge channel arranged at the front end around the viewing channel, and a water spray channel and an air jet channel arranged at the rear end around the viewing channel; the water spray channel and the air jet channel together communicate with the same discharge channel;
the axis of the viewing channel is collinear with the axis of the ear insertion tube; the front end of the discharge channel faces the gap between the viewing tube and the ear insertion tube; the rear end of the water spray channel is connected to the end of the water spray tube communicating with the interior of the ear insertion tube; the rear end of the air jet channel is connected to the end of the air tube communicating with the interior of the ear insertion tube.

7. The ear canal cleaning device according to claim 4, wherein,
the handle is tubular, and at least one pipeline separation ring is provided inside the handle;
the outer ring wall of the pipeline separation ring is adapted to the inner wall of the handle; inside the pipeline separation ring, an air tube separation ring for passing the air tube, a water spray tube separation ring for passing the water spray tube, a water suction tube separation ring for passing the water suction tube, and a wire separation ring for passing wires are separated by an isolation bar and a separation outer ring;
the air tube separation ring, the water spray tube separation ring, and the water suction tube separation ring are all annular, with their centers collinear, and are arranged in the middle of the pipeline separation ring, so that a wire separation ring is formed between these three rings and the separation outer ring of the pipeline separation ring.

8. The ear canal cleaning device according to claim 4, further comprising: at least one air path heating device;
the air path heating device includes an air path channel tube and an air path heating wire wound around the outside of the air path channel tube; the air path heating wire is electrically connected to the control module and controlled by the control module to adjust its heat output;
the air tube includes at least two sub-air tubes, and both ends of the air path channel tube are connected between and communicate with two adjacent sub-air tubes, so that the air path heating device is installed within the pipeline of the air tube; controlled by the control module, the air path heating wire heats the air flowing through the air path channel tube.

9. The ear canal cleaning device according to claim 4, wherein,
the water tank includes a clean water tank for storing cleaning water for flushing the ear canal and a wastewater tank for storing wastewater sucked back by the water suction tube after flushing;
the water spray pump pumps water from the clean water tank to the water spray tube; the water suction pump draws water from the water suction tube to the wastewater tank;
a water path heating wire is installed inside the clean water tank, which is electrically connected to the control module and controlled by the control module to adjust its heat output;
controlled by the control module, the water path heating wire heats the cleaning water pumped into the water spray tube.

10. The ear canal cleaning device according to claim 1, wherein,
the top of the base is provided with a sunken wire accommodating cavity with a cylindrical surrounding wall;
a wire winding base is provided in the middle of the wire accommodating cavity; the top of the wire winding base is provided with a sunken handle cavity with a cylindrical surrounding wall; the side wall of the wire winding base is provided with a wire outlet, which is a sunken notch penetrating the side wall of the wire winding base; a wire winding cavity is formed between the inner wall of the wire accommodating cavity and the outer wall of the wire winding base for leading out wires connecting components inside the base and components inside the handheld component from the handle cavity; when the handheld component is inserted into the base, the wires enter the wire winding cavity from the wire outlet and are wound around the outer wall of the wire winding base, and the handle of the handheld component is inserted into the handle cavity of the wire winding base;
the top edge of the wire winding base extends outward to form a wire-blocking top eave, which prevents the wires wound around the wire winding base from disengaging from the top of the wire winding base.

11. The ear canal cleaning device according to claim 4, wherein,
the top of the base is provided with a sunken wire accommodating cavity with a cylindrical surrounding wall;
a wire winding base is provided in the middle of the wire accommodating cavity; the top of the wire winding base is provided with a sunken handle cavity with a cylindrical surrounding wall; the side wall of the wire winding base is provided with a wire outlet, which is a sunken notch penetrating the side wall of the wire winding base; a wire winding cavity is formed between the inner wall of the wire accommodating cavity and the outer wall of the wire winding base for leading out wires connecting components inside the base and components inside the handheld component from the handle cavity; when the handheld component is inserted into the base, the wires enter the wire winding cavity from the wire outlet and are wound around the outer wall of the wire winding base, and the handle of the handheld component is inserted into the handle cavity of the wire winding base;
the top edge of the wire winding base extends outward to form a wire-blocking top eave, which prevents the wires wound around the wire winding base from disengaging from the top of the wire winding base.
